(19) United States
(12) Patent Application Publication
(10) Pub. No.: US 2009/0010887 A1
(43) Pub. Date: Jan. 8, 2009

(54) Method and composition for enhancing anti-angiogenic therapy

(76) Inventor: Shmuel A. Ben-Sasson, Jerusalem (IL)

Correspondence Address: DAVID S. RESNICK, NIXON PEABODY LLP, 100 SUMMER STREET, BOSTON, MA, 02110-2131, US

(21) Appl. No.: 11/664,957

(22) PCT Filed: Oct. 5, 2005
(86) PCT No.: PCT/IB05/04069

§ 371 Date: Apr. 6, 2007

Related U.S. Application Data
(60) Provisional application No. 60/616,348, filed on Oct. 6, 2004.

(51) Int. Cl. A61K 31/664; A61K 31/195; A61K 31/122; A61K 31/44; A61K 39/395; A61K 31/74; A61K 38/21; A61K 31/56; A61K 31/606; A61P 3/00; A61P 17/06; A61P 35/00; A61P 27/02; A61P 19/02

(52) U.S. Cl. 424/85.7; 514/110; 514/567; 514/682; 514/352; 424/142.1; 424/78.17; 424/158.1; 514/171; 514/166

(57) ABSTRACT

The present invention relates to the surprising discovery that agents that increase intracellular accumulation of NADH+H⁺ enhance the anti-cancer effects of angiogenesis inhibitors. Furthermore, treatment of a mammal with a combination of at least one angiogenesis inhibitor and at least one agent that enhances intracellular accumulation of NADH+H⁺ allows for the enhanced treatment and/or prevention of angiogenic diseases and disorders.

### CROSS REFERENCE TO RELATED APPLICATIONS

The present application claims benefit under 35 U.S.C. 119(e) of U.S. Provisional Application No. 60/616,348, filed Oct. 6, 2004, the contents of which are incorporated herein by reference in their entirety.

### BACKGROUND OF THE INVENTION

Cancer generally refers to one of a group of more than 100 diseases caused by the uncontrolled, abnormal growth of cells that can spread to adjoining tissues or other parts of the body. Cancer cells can form a solid tumor, in which the cancer cells are massed together, or exist as dispersed cells, as in leukemia. Normal cells divide until maturation is attained and then only as necessary for replacement of damaged or dead cells. Cancer cells are often referred to as “malignant”, because they divide endlessly, eventually crowding out nearby cells and spreading to other parts of the body. The tendency of cancer cells to spread from one organ to another or from one part of the body to another distinguishes them from benign tumor cells, which overgrow but do not spread to other organs or parts of the body. Malignant cancer cells eventually metastasize and spread to other parts of the body via the bloodstream or lymphatic system, where they can multiply and form new tumors. This sort of tumor progression makes cancer a deadly disease.

Although there have been great improvements in the diagnosis and treatment of cancer, many people die from cancer each year, and their deaths are typically due to metastases and cancers that are resistant to conventional therapies.

Most drug-mediated cancer therapies rely on poisons, called cytotoxic agents, selective for dividing cells. These drugs are effective because cancer cells generally divide more frequently than normal cells. However, such drugs almost inevitably do not kill all of the cancer cells in the patient. One reason is that cancer cells can acquire mutations that confer drug resistance. Another is that not all cancer cells divide more frequently than normal cells, and slowly-dividing cancer cells can be as, or even more, insensitive to such cytotoxic agents as normal cells. Some cancer cells divide slowly, because they reside in a poorly vascularized, solid tumor and are unable to generate the energy required for cell division. As a tumor grows, it requires a blood supply and, consequently, growth of new vasculature.

Angiogenesis is a process of tissue vascularization that involves the growth of new developing blood vessels into a tissue, and is also referred to as neo-vascularization. Blood vessels are the means by which oxygen and nutrients are supplied to living tissues and waste products are removed from living tissue. When appropriate, angiogenesis is a critical biological process. For example, angiogenesis is essential in reproduction, development and wound repair. Conversely, inappropriate angiogenesis can have severe negative consequences. For example, it is only after solid tumors are vascularized as a result of angiogenesis that the tumors have a sufficient supply of oxygen and nutrients that permit it to grow rapidly and metastasize.

Angiogenesis-dependent diseases are those diseases which require or induce vascular growth. Such diseases represent a significant portion of all diseases for which medical treatment is sought, and include obesity, inflammatory disorders such as immune and non-immune inflammation, chronic articular rheumatism and psoriasis, disorders associated with inappropriate or inopportune invasion of vessels such as macular degeneration, diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

The therapeutic implications of pro-angiogenic factors were first described by Folkman and colleagues over three decades ago (Folkman, N. Engl. J. Med., 285:1182-1186 (1971)). Abnormal angiogenesis occurs when the body loses at least some control of angiogenesis, resulting in either excessive or insufficient blood vessel growth. For instance, conditions such as ulcers, strokes, and heart attacks may result from the absence of angiogenesis normally required for natural healing. In contrast, excessive blood vessel proliferation can result in tumor growth, tumor spread, obesity, macular degeneration, blindness, psoriasis and rheumatoid arthritis.

Angiogenesis is a multifaceted process. Direct angiogenesis inhibitors prevent vascular endothelial cell growth. Indirect angiogenesis inhibitors prevent the activation of angiogenesis or block the expression of receptors that aid in the onset of angiogenesis. Angiogenesis inhibitors have shown promise in animal studies and clinical trials are currently underway (Kerbel et al. Nature Reviews, Vol. 2, pp. 727-739). However, angiogenesis inhibitors have not proven 100% effective for all cancers.

The treatment of cancer has thus far proved problematic. While “cancers” share many characteristics, each particular cancer has its own specific characteristics. Genetics and environmental factors have a complex interplay in the severity and prognosis of treatment. Thus, treatment must be carefully tailored.

Although cancer chemotherapy has advanced dramatically in recent years, treating cancers with a single agent has had limited success. First, any single agent may only target a subset of the total population of malignant cells present, leaving a subpopulation of cancerous cells to continue growing. Second, cells develop resistance upon prolonged exposure to a drug. Combination therapies, which employ two or more agents with differing mechanisms of action and differing toxicities, have been useful for circumventing drug resistance and increasing the target cell population, but have not proven effective in the treatment of all cancers. In addition, certain combinations of agents may be synergistic: their combined effect is larger than that predicted based on their individual activities. Thus, combining different agents can be a powerful strategy for treating cancer.

However, combination therapies are a hit or miss proposition. In many cases, cross effects and treatment load can result in lower effectiveness for the combination than either treatment alone. Multidrug resistance can also be a problem.

Cytotoxic agents such as cyclophosphamide have also been used to treat cancers. The most striking difference between malignant and healthy cells is the capacity of cancer cells for unrestricted proliferation. This difference is exploited by many cytotoxic agents, which typically disrupt cell proliferation by interfering with the synthesis or integrity of DNA. Examples of classes of cytotoxic agents which function in this manner include alkylating agents, antimetabolites (e.g. purine and pyrimidine analogues), and platinum coordination complexes.

One problem with cytotoxic agents which function by disrupting cell division is that they don't discriminate between normal and malignant cells: any dividing cell is a potential target for their action. Thus, cell populations which normally exhibit high levels of proliferation (such as bone marrow) are targeted, leading to the toxic side effects commonly associated with cancer treatments.

Inhibitors of pro-angiogenic growth factors are agents used to inhibit the signaling of known pro-angiogenic factors like VEGF or FGF. Such agents can act extracellularly, by the inhibition of the interaction of an angiogenic factor with its receptor or can act intracellularly via the inhibition of the protein-kinase activity of the corresponding receptors. These agents include, for example, anti-VEGF or anti-VEGF-Receptor antibodies or inhibitors of the protein-kinase domain of VEGF-R, FGF-R or PDGF-R. Currently, these agents by themselves failed to demonstrate sufficient efficacy in the treatment of cancer.

With only a few exceptions, no single drug or drug combination is curative for most cancers. Thus, new drugs or combinations that can delay the growth of life-threatening tumors and/or improve quality of life by further reducing tumor load are needed.

### SUMMARY OF THE INVENTION

The present invention is directed to a method of inhibiting angiogenesis in a tissue of a mammal having an angiogenic disease or disorder or is at risk for developing an angiogenic disease or disorder comprising administering to a mammal at least one angiogenesis-inhibitor in combination with at least one agent that enhances NADH+H⁺ production. Such agents include, for example, alcohols or poly-alchohols (polyols).

In one embodiment of the present invention the angiogenesis inhibitor is a direct angiogenesis inhibitor (i.e. Avastin). In another embodiment, the angiogenesis inhibitor is an indirect angiogenesis inhibitor (i.e. ZD1839 (Iressa)). In a further embodiment, the angiogenesis inhibitor is an anti-inflammatory agent such as diclofenac, indomethacin, sulfasalazine, CELEBREX® (Celecoxib), THALOMID® (Thalidomide), or IFN-α, or a redox quinone such as, for example, menadione, or a cytotoxic agent such as, for example, low dose cyclophosphamide.

In a preferred embodiment, the agent that enhances NADH+H⁺ production is a poly-alcohol. The poly-alcohol is most preferably xylitol. Alternatively, the poly-alcohol is mannitol, sorbitol, arabinol and iditol. Furthermore, the present invention is directed to method of inhibiting angiogenesis in a tissue of a mammal having an angiogenic disease or disorder such as cancer.

In another embodiment of the present invention, the methods are directed to the treatment of a solid tumor or solid tumor metastasis.

In yet another embodiment, the methods are directed to the treatment of retinal tissue and said disease or disorder is retinopathy, diabetic retinopathy, or macular degeneration. Alternatively, the methods of the present invention are directed toward treatment of tissue at risk of restenosis, wherein the tissue is at the site of coronary angioplasty.

In another embodiment of the present invention, the methods are directed toward inhibiting angiogenesis in a tissue of a mammal, wherein said tissue is inflamed and said disease or disorder is arthritis or rheumatoid arthritis. Alternatively, such mammal tissue is adipose tissue and said disease is obesity.

The methods of the present invention can be used either alone, or in conjunction with other treatment methods known to those of skill in the art. Such methods may include, but are not limited to, radiation therapy or surgery.

The mammal to be treated by the methods of the present invention may include a human or a domestic animal, such as a cat or dog.

In another embodiment of the present invention, said administering comprises intravenous, transdermal, intrasynovial, intramuscular, or oral administration.

In a further embodiment of the present invention, said orally administered composition is an aqueous suspension or solution that might further contain a flavoring agent (i.e. menthol and/or anethol). Some constituents of such aqueous suspension or solution might be supplied in a dry form and reconstituted (=solubilized) shortly prior to oral administration.

The methods of the present invention allow for the administration of at least one angiogenesis inhibitor and an agent that enhances intracellular accumulation of NADH+H⁺ either prophylactically or therapeutically.

The methods of the present invention further allow for a weekly cycle of the administration of the at least one angiogenesis inhibitor and an agent that enhances intracellular accumulation of NADH+H⁺. Such a cycle may include twice a week administration of said combination on non-consecutive days, while an agent that enhances intracellular accumulation of NADH+H⁺ and a redox quinone only are administered daily during the rest of the week. A detailed description of such alternated treatment is provided in Example 1 below.

The methods of the present invention are directed toward inhibiting an angiogenic disease or disorder in a mammal at risk for developing an angiogenic disease or disorder. The risk can be determined utilizing genetic tools. Alternatively, the risk can be determined by measuring levels of cancer marker proteins in the biological fluids (i.e. blood, urine) of a patient. Marker proteins include, for example, calcitonin, PSA, CEA, thymosin β-15, thymosin β-16, and matrix metalloproteinase (MMP).

In another embodiment, the invention provides a pharmaceutical composition comprising a combination of at least one angiogenesis inhibitor, at least one agent that enhances NADH+H⁺ production, such as, for example, a polyol, and a pharmaceutically acceptable carrier.

In a preferred embodiment, the at least one angiogenesis inhibitor is the composition described in U.S. application Ser. No. 10/898,721, incorporated herein by reference. This inhibitor comprises a cytotoxic agent, preferably cyclophosphamide, an anti-inflammatory agent, preferably a COX1-2 inhibitor such as diclofenac and indomethacin, and a redox quinone, preferably Vitamin K₃ (or menadione or menadione sodiumbisulfite). An ester of benzoic acid, preferably Benzyl benzoate can also be included. In addition to this preferred angiogenesis inhibitor, the composition of the present invention comprises an agent that enhances intracellular accumulation of NADH+, such as, for example, a polyol, preferably xylitol, and a pharmaceutically acceptable carrier. An NFκB inhibitor, such as sulfasalazine, preferably should be included.

In certain embodiments, the combination further includes a bisphosphonate, preferably pamidronate or alendronate. In other embodiments, the combination further includes a matrix metalloproteinase (MMP) inhibitor.

As used herein, a “cytotoxic agent” acts as an angiogenesis inhibitor when administered at a low dose. Preferred cytotoxic agents include, for example, cyclophosphamide, ifosfamide, cytarabine, 6-mercaptopurine, 6-thioguanine, vincristine, doxorubicin, and daunorubicin, chlorambucil, carmustine, vinblastine, methotrexate, and paclitaxel. More preferred cytotoxic agents include cyclophosphamide, ifosfamide, cytarabine, 6-mercaptopurine, 6-thioguanine, vincristine, mitoxantrone, doxorubicin, and daunorubicin. Cyclophosphamide and ifosfamide are most preferred cytotoxic agents.

The angiogenesis inhibitor may also be an inhibitor of pro-angiogenic growth factors. As used herein, the phrase “inhibitors of pro-angiogenic growth factors” means agents used to inhibit the signaling of known pro-angiogenic factors like VEGF or FGF. Such agents can act extracellularly, by the inhibition of the interaction of an angiogenic factor with its receptor or can act intracellularly via the inhibition of the protein-kinase activity of the corresponding receptors. These agents include, for example, anti-VEGF or anti-VEGF-Receptor antibodies (U.S. Pat. No. 6,416,758 and WO 01/72829) or inhibitors of the protein-kinase domain of VEGF-R, FGF-R or PDGF-R (WO 97/34876 and U.S. Pat. No. 6,462,060).

The pharmaceutical composition of the present invention may also comprise a matrix metalloproteinase (MMP) inhibitor. As used herein, the phase “matrix metalloproteinase (MMP) inhibitor” means any chemical compound that inhibits by at least five percent the hydrolytic activity of at least one matrix metalloproteinase enzyme that is naturally occurring in a mammal. Such compounds are also referred to as “MMP inhibitors”.

Numerous matrix metalloproteinase inhibitors are known, and all are useful in the present invention. Some specific examples of MMP inhibitors useful in the present invention are AG-3340, RO 32-3555, RS 13-0830, Tissue Inhibitors of Metalloproteinases (TIMPs) (e.g. TIMP-1, TIMP-2, TIMP-3, or TIMP4), alpha 2-macroglobulin, tetracyclines (e.g., tetracycline, minocycline, and doxycycline), hydroxamates (e.g. BATIMASTAT, MARIMISTAT and TROCADE), chelators (e.g., EDTA, cysteine, acetylcysteine, D penicillamine, and gold salts), synthetic MMP fragments, succinyl mercaptopurines, phosphonamidates, and hydroxaminic acids.

In one embodiment, said pharmaceutical composition is formulated in the form of an aqueous suspension or a solution ready for oral administration. Some constituents of such aqueous suspension or solution might be supplied in a dry form and reconstituted (=solubilized) shortly prior to oral administration.

In another embodiment, such formulation further contains a flavoring agent (e.g. menthol and/or anethol).

In yet further embodiment, a separate formulation is prepared that contains only an enhancer of intracellular NADH+H⁺ accumulation (e.g. xylitol) together with a redox quinone (e.g. menadione) and, preferably, an NFκB inhibitor, such as sulfasalazine and a flavoring agent (e.g. menthol and/or anethol).

The present invention also includes a kit having components of the combination and directions for their administration.

### BRIEF DESCRIPTION OF THE DRAWINGS

FIG. 1: Xylitol Improves Anti-Cancer Efficacy of Anti-angiogenic Therapy. FIG. 1 shows tumor volume (mm³) for three groups of mice harboring cyclophosphamide-resistant Breast cancer tumors. Five days after tumor-cell inoculation the group treated with the anti-angiogenic therapy (hereby referred to as 4×4) and the group treated with 4×4+Xylitol, both received cyclophosphamide plus diclofenac plus menadione doses i.p. twice a week over the four weeks following inoculation. Doses without cyclophosphamide plus diclofenac were given on the remaining 4 days of the week over the same period of time. For details see Table 1 below. The control group received only the vehicle (2% Pluronic, 2% Solutol HS-15 in DDW) i.p. 6 days a week over the four weeks. The tumor volume of mice given the 4×4 combination plus xylitol was greatly reduced as compared to control mice (4×4 only or vehicle controls.)

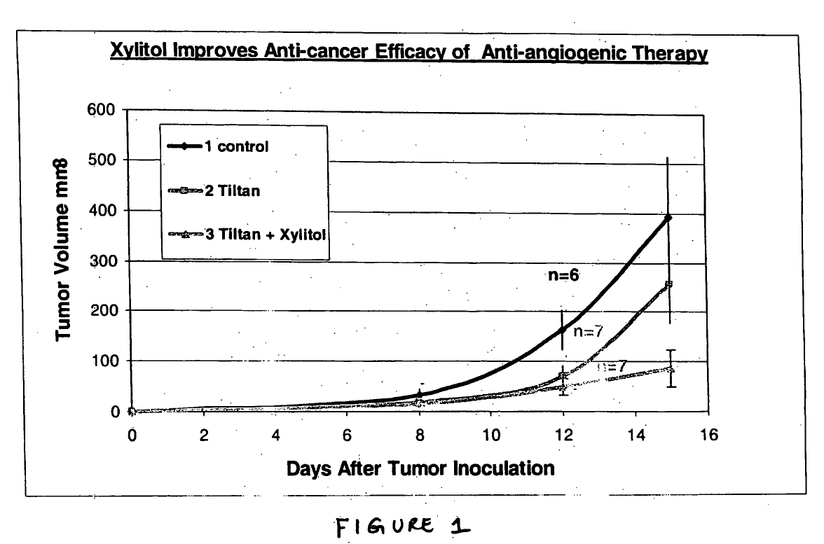

FIG. 2 shows the mean tumor volume (mm³) of tumor-bearing mice that received either control, Tiltan and Sulfasalazine Treatment (as described in Example 2; TB002) as a function of time after inoculation (n=7-8 mice per group; SE)

FIG. 3 shows the mean tumor volume (mm³) of tumor-bearing mice that received either control, Tiltan and Sulfasalazine Treatment (as described in Example 2; TB004) as a function of time after inoculation (n=7-8 mice per group; SE)

FIG. 4 shows that following treatment initiation (described in Example 3), both tumor markers CA-125 and CA-15.3 dropped to the normal range level and stayed at this range through wk 30.

FIG. 5 shows that after 6 wks on the TiltAn treatment, a CT of the pelvis, abdomen and thorax revealed stable disease. On wk 12 there was a decrease in the dimensions of the liver metastasis and this decrease proceeded through wk 30.

### DETAILED DESCRIPTION OF THE INVENTION

We have found that agents which increase intracellular accumulation of NADH+H⁺ (i.e. poly-alcohols or polyols) enhance the effect of angiogenesis inhibitors, i.e. their anti-cancer effect. As such, the present invention is directed to methods for inhibiting angiogenesis in a tissue of a mammal having an angiogenic disease or disorder or at risk for developing an angiogenic disease or disorder by administering an effective amount of at least one agent that enhances intracellular accumulation of NADH+H⁺ in combination with at least one angiogenesis inhibitor.

Also encompassed in the present invention are pharmaceutical compositions comprising at least one angiogenesis inhibitor, at least one agent that enhances intracellular accumulation of NADH+H⁺ and a pharmaceutically acceptable carrier.

In one embodiment of the present invention the angiogenesis inhibitor is a direct angiogenesis inhibitor (i.e. Angiostatin, Bevacizumab (Avastin), Arresten, Canstatin, Combretastatin, Endosiatin, NM-3, Thrombospondin, Tumstatin, 2-methoxyestradiol, and Vitaxin). Alternatively, the angiogenesis inhibitor may be an indirect angiogenesis inhibitors (i.e. ZD1839 (Iressa), ZD6474, OS1774 (Tarceva), C11033, PK11666, IMC225 (Erbitux), PTK787, SU6668, SU11248, Herceptin, TNP-470, HPMA co-polymer-TNP-470 and IFN-α).

In the context of the present application, angiogenesis inhibitors also include cytotoxic agents. Cytotoxic agents are used to treat abnormal and uncontrolled progressive cellular growth. Examples include the alkylating agents cyclophosphamide (Bristol-Meyers Squibb), ifosfamide (Bristol-Meyers Squibb), chlorambucil (Glaxo Wellcome), and carmustine (Bristol-Meyers Squibb); the anti-metabolites cytarabine (Pharmacia & Upjohn), 6-mercaptopurine (Glaxo Wellcome), 6-thioguanine (Glaxo Wellcome), and methotrexate (Immunex); the antibiotics doxorubicin (Pharmacia & Upjohn), daunorubicin (NeXstar), and mitoxantrone (Immunex); and miscellaneous agents such as vincristine (Lilly), vinblastine (Lilly), and paclitaxel (Bristol-Meyers Squibb). Preferred cytotoxic agents include cyclophosphamide, ifosfamide, cytarabine, 6-mercaptopurine, 6-thioguanine, doxorubicin, daunorubicin, mitoxantrone, and vincristine. The most preferred cytotoxic agent is cyclophosphamide and ifosfamide.

The angiogenesis inhibitor may also be an inhibitor of pro-angiogenic growth factors. Such agents are used to inhibit the signaling of known pro-angiogenic factors like VEGF or FGF. Such agents can act extracellularly, by the inhibition of the interaction of an angiogenic factor with its receptor or can act intracellularly via the inhibition of the protein-kinase activity of the corresponding receptors. These agents include, for example, anti-VEGF or anti-VEGF-receptor antibodies (U.S. Pat. No. 6,416,758 and WO 01/72829) or inhibitors of the protein-kinase domain of VEGF-R, FGF-R or PDGF-R (WO 97/34876 and U.S. Pat. No. 6,462,060).

CELEBREX® (Celecoxib), THALOMID® (Thalidomide), and IFN-α have also been recognized as angiogeneis inhibitors (Kerbel et al., Nature Reviews, Vol. 2, October 2002, pp. 727) and are encompassed in the methods and compositions of the present invention. However, it is noteworthy that while some of these inhibitors are anti-inflammatory agents, they are distinct from the preferred anti-anflammatory agents according to the present invention. Thus, while Celecoxib is an exlusive COX2, but not COX1, inhibitor and Thalidomide is an attenuator of TNFα response, the preferred agents according to the present invention are COX1-2 inhibitors such as diclofenac or indomethacin, and NFκB inhibitors, such as sulfasalazine.

In a preferred embodiment, the angiogenesis inhibitor is the composition described in U.S. application Ser. No. 10/898,721, incorporated herein by reference. The angiogenesis inhibitors of U.S. Ser. No. 10/898,721 comprise a cytotoxic agent, preferably cyclophosphamide, an anti-inflammatory agent, preferably a COX1-2 inhibitor such as diclofenac and indomethacin, a redox quinone, preferably Vitamin K₃ (or menadione or menadione sodiumbisulfite) and a pharmaceutically acceptable carrier. An ester of benzoic acid, preferably Benzyl benzoate can also be included.

We have found that agents which increase intracellular accumulation of NADH+H⁺ (i.e. poly-alcohols or polyols) enhance the anti-cancer effect of angiogenesis inhibitors. Thus, in addition to the at least one angiogenesis inhibitor, the composition and methods of the present invention further comprise at least one agent which increase intracellular accumulation of NADH+H⁺. In a preferred embodiment, the NADH+H⁺ increasing agent is a poly-alcohol (polyol). In a most preferred embodiment, the polyol is xylitol.

Alternatively, the poly-alcohol is mannitol, sorbitol, arabinol, iditol or any other polyol known to those of skill in the art.

In certain embodiments, the combination further includes a bisphosphonate, preferably pamidronate or alendronate. In other embodiments, the combination further includes a matrix metalloproteinase (MMP) inhibitor. As used herein, the phase “matrix metalloproteinase (MMP) inhibitor” means any chemical compound that inhibits by at least five percent the hydrolytic activity of at least one matrix metalloproteinase enzyme that is naturally occurring in a mammal. Such compounds are also referred to as “MMP inhibitors”.

Numerous matrix metalloproteinase inhibitors are known, and all are useful in the present invention. Some specific examples of MMP inhibitors useful in the present invention are AG-3340, RO 32-3555, RS 13-0830, Tissue Inhibitors of Metalloproteinases (TIMPs) (e.g. TIMP-1, TIMP-2, TIMP-3, or TIMP-4), alpha 2-macroglobulin, tetracyclines (e.g., tetracycline, minocycline, and doxycycline), hydroxamates (e.g. BATIMASTAT, MARIMISTAT and TROCADE), chelators (e.g., EDTA, cysteine, acetylcysteine, D penicillamine, and gold salts), synthetic MMP fragments, succinyl mercaptopurines, phosphonamidates, and hydroxaminic acids.

In additional embodiments, the combination further includes an NFκB inhibitor, preferably sulfasalazine. In yet additional embodiments, the combination further includes a separate composition of the intracellular NADH+H⁺-increasing agent together with a redox quinone, preferably Vitamin K₃ K₃ and an NFκB inhibitor, such as sulfasalazine. This composition is administered on days where the other angiogenesis inhibitors are not given.

The present invention is directed to method of inhibiting angiogenesis in a tissue of a mammal having an angiogenic disease or disorder such as cancer. The cancer may include, but is not limited to, lung cancer (e.g. adenocarcinoma and including non-small cell lung cancer), pancreatic cancers (e.g. pancreatic carcinoma such as, for example exocrine pancreatic carcinoma), colon-cancers (e.g. colorectal carcinomas, such as, for example, colon adenocarcinoma and colon adenoma), prostate cancer including the advanced disease, hematopoietic tumors of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumors of mesenchymal origin (e.g. fibrosarcomas and rhabdomyosarcomas), melanomas, teratocarcinomas, neuroblastomas, gliomas, benign tumor of the skin (e.g. keratoacanthomas), breast carcinoma (e.g. advanced breast cancer), kidney carcinoma, ovary carcinoma, bladder carcinoma and epidermal carcinoma.

The methods of the present invention may be directed to the treatment of a solid tumor or solid tumor metastasis.

In yet another embodiment, the methods are directed to the treatment of retinal tissue and said disease or disorder is retinopathy, diabetic retinopathy, or macular degeneration. Alternatively, the methods of the present invention are directed toward treatment of tissue at risk of restenosis, wherein the tissue is at the site of coronary angioplasty.

In another embodiment of the present invention, the methods are directed toward inhibiting angiogenesis in a tissue of a mammal, wherein said tissue is inflamed and said disease or disorder is arthritis or rheumatoid arthritis. Alternatively, said tissue is an adipose tissue and said disease is obesity.

The combination therapy of the present invention can be used either alone, or in conjunction with other treatment methods known to those of skill in the art. Such methods may include, but are not limited to radiation therapy or surgery.

The angiogenesis inhibitor and agent which increases intracellular accumulation of NADH+H⁺ of the present invention can be administered via any medically acceptable means which is suitable for the compounds to be administered, including oral, rectal, topical, transdermal, intrasynovial, intramuscular or parenteral (including subcutaneous, intramuscular and intravenous) administration. The pharmaceutical combination or each agent individually can be administered by any means known in the art. Such modes include oral, rectal, nasal, topical (including buccal and sublingual), or parenteral (including subcutaneous, intramuscular, intravenous, and intradermal) administration, including sustained release formulations.

For ease to the patient, oral administration is preferred and in such a case a flavoring agent (i.e. menthol) might be added. However, typically oral administration requires a higher dose than an intravenous administration. Thus, administration route will depend upon the situation: the skilled artisan must determine which form of administration is best in a particular case, balancing dose needed versus the number of times per month administration is necessary.

In administering the compounds one can use the normal dose of each compound individually. However, if the angiogenesis inhibitor is a cytotoxic agent, in order to reduce-side effects, preferably one uses a lower level than used when given as a single cytotoxic agent—typically 75% or less of the individual amount, more preferably 50% or less, still more preferably 40% or less. Preferably, the agent that enhances intracellular accumulation of NADH+H⁺ (i.e. polyol) is given at a dose of 5 g to 100 g per day, most preferably at a dose of 10 g to 50 g per day.

The angiogenesis inhibitors may be administered in any manner found appropriate by a clinician, such as those described for individual cytotoxic agents in the PDR. For example, when the cytotoxic agent in cyclophosphamide, the dose is preferably 0.1-50 mg/kg, most preferably 0.2-20 mg/kg.

As with the use of other chemotherapeutic drugs, the individual patient will be monitored in a manner deemed appropriate by the treating physician. Typically, no additional drug treatments will occur until, for example, the patient's neutrophil count is at least 1500 cells/mm³. Dosages can also be reduced if severe neutropenia or severe peripheral neuropathy occurs, or if a grade 2 or higher level of mucositis is observed, using the Common Toxicity Criteria of the National Cancer Institute.

In therapeutic applications, the dosages and administration schedule of the agents used in accordance with the invention vary depending on the agent, the age, Weight, and clinical condition of the recipient patient, and the experience and judgment of the clinician or practitioner administering the therapy, among other factors affecting the selected dosage. Generally, the dose and administration scheduled should be sufficient to result in slowing, and preferably regressing, the growth of the tumor(s) and also preferably causing complete regression of the cancer. In some cases, regression can be monitored via direct imaging (e.g. MRI) or by a decrease in blood levels of tumor specific markers. An effective amount of a pharmaceutical agent is that which provides an objectively identifiable improvement as noted by the clinician or other qualified observer. Regression of a tumor in a patient is typically measured with reference to the diameter of a tumor. Decrease in the diameter of a tumor indicates regression. Complete regression is also indicated by failure of tumors to reoccur after treatment has stopped.

The agents in combination, or separately, are delivered at periodic intervals that can range from several times a day to once per month. As noted above, the agents are administered until the desired therapeutic outcome has been obtained. Additionally, in order to avoid side-effects not all components of the combination need to be delivered at each administration. For example, the xylitol and menadione may be delivered everyday, whereas the other angiogenesis inhibitors (i.e. cyclophosphamide and diclofenac) may be delivered twice a week.

The methods of the present invention allow for the administration of the angiogenesis inhibitor(s) and intracellular NADH+H⁺ increasing agent either prophylactically or therapeutically.

When provided prophylactically, the compounds are provided in advance of any symptom. The prophylactic administration of the compounds serves to prevent or inhibit an angiogenesis disease or disorder, i.e. cancer. Prophylactic administration of the agent which increases intracellular accumulation of NADH+H⁺ and angiogenesis inhibitor may be given to a patient with, for example, a family history of cancer. Alternatively, administration of the compounds of the invention may be given to a patient with rising cancer marker protein levels. Such markers include, for example, rising PSA, CEA, thymosin β-15, thymosin β-16, calcitonin, and matrix metalloproteinase (MMP). When provided prophylactically, the dose of either the angiogenesis inhibitor(s), agent which increases accumulation of NADH+H⁺, or both may be reduced appropriately.

When provided therapeutically, the compounds are provided at (or after) the onset of a symptom or indication of an angiogenesis disease or disorder. Thus, the combination therapy of the present invention may be provided either prior to the anticipated angiogenesis at a site or after the angiogenesis has begun at a site.

The methods of the present invention are directed toward inhibiting an angiogenic disease or disorder in a mammal at risk for developing an angiogenic disease or disorder. The risk can be determined utilizing genetic tools. Alternatively, the risk can be determined by measuring levels of cancer marker proteins in the biological fluids (i.e. blood, urine) of a patient. Marker proteins include, for example, calcitonin, PSA, CEA, thymosin β-15, thymosin β-16, and matrix metalloproteinase (MMP).

In a related embodiment, the invention contemplates the practice of the method in conjunction with other therapies such as chemotherapy, radiation therapy, or surgery. In one embodiment, the methods are directed against solid tumors and for control of establishment of metastases. The administration of angiogenesis-inhibiting amounts of at least one agent that increases accumulation of NADH+H⁺ and at least one anti-angiogenic compound may be conducted before, during or after other therapies. In addition, the compounds of the present invention may be administered concurrently with other cancer therapies known to those of skill in the art.

Insofar as the present methods apply to inhibition of tumor neovascularization, the methods can also apply to inhibition of tumor tissue growth, to inhibition of tumor metastases formation, and to regression of established tumors.

### Pharmaceutical Compositions

In yet a further embodiment of the present invention, we provide a pharmaceutical composition comprising a combination of at least one angiogenesis inhibitor, at least one agent which enhances intracellular accumulation of NADH+H⁺ and a pharmaceutically acceptable carrier.

In on embodiment, the composition includes a controlled-release device where one or several of the drugs are being released in a delayed fashion. Such formulation can be in the form of a tablet (or a pill) which releases different doses of drugs in different time intervals after being taken orally.

The pharmaceutical compositions of this invention which are found in combination may be in the dosage form of solid, semi-solid, or liquid such as, e.g. suspension, aerosols, or the like. Preferably the compositions are administered in unit dosage forms suitable for single administration of precise dosage amounts. The compositions may also include, depending on the formulation desired, pharmaceutically-acceptable, nontoxic carriers or diluents, which are defined as vehicles commonly used to formulate pharmaceutical compositions for animal or human administration.

Compositions may be provided as sustained release or timed release formulations. The carrier or diluent may include any sustained release material known in the art, such as glyceryl monostrearate or glyceryl distearate, alone or mixed with a wax. Microencapsulation may also be used. The timed release formulation can provide a combination of immediate and pulsed release throughout the day. The diluent is selected so as not to affect the biological activity of the combination. Examples of such diluents are distilled water, physiological saline, Ringer's solution, dextrose solution, and Hank's solution. In addition, the pharmaceutical composition of formulation may also include other carriers, adjuvants, emulsifiers such as poloxamers, or nontoxic, nontherapeutic, nonimmunogenic stabilizers and the like. Effective amounts of such diluent or carrier will be those amounts which are effective to obtain a pharmaceutically acceptable formulation in terms of solubility of components, or biological activity, and the like.

Kits for the inhibition of angiogenesis are also encompassed in the present invention. The kits comprise at least one vial of an angiogenesis inhibitor, at least one vial of an agent which increases intracellular accumulation of NADH+H⁺ and a pharmaceutical carrier. Most preferably, the kit contains instructions describing their use in combination.

Accordingly, the present invention relates to an antineoplastic/anti-angiogenic combination of at least two agents, and to a method for treating angiogenic diseases or disorder, i.e. cancer, macular degeneration or obesity.

Advantageously, all agents of said combination are formulated in a single dosage form that is preferably administered once a day. It is further advantageous to provide said oral formulation in a liquid form.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications, and publications cited herein are incorporated herein by reference.

### EXAMPLE 1

Cyclophosphaide-resistant Breast cancer cells of the EMT-6/CTX cell line were thawed, grown in tissue culture plates and injected (10⁶ cells/ml) s.c. into the posterior flank of male 27 g CB6F1 mice.

The anti-angiogenic treatment (hereby defined as “4×4”) comprises a cyclical combination of drugs as detailed in Table 1. The efficacy of the 4×4 treatment with xylitol (group #3) or without it (group #2), was compared.

The mice were divided into three groups of seven. Five days after tumor inoculation the 4×4 group and the 4×4+Xylitol group received the corresponding doses i.p. (mg/Kg doses for each group are indicated in Table 1). The doses that contain menadione, cyclophosphamide and diclofenac (=full combination) were given twice a week (Sundays and Wednesdays) over the four weeks following inoculation, while the menadione-only doses were given on the remaining 4 days of the week (all but Saturday), over the same period of time. The control group received only the vehicle¹ i.p. 6 days a week over the four weeks.

TABLE 1
Group
No.

| 1 | Control | Vehicle¹ | | | | |
|---|---|---|---|---|---|---|
| | 6 days/week | | | | | |
| 2 | 4X4 | Cyclophosphamide | Diclofenac | Menadione | 4X4 | Menadione |
| | 2 non- | in vehicle¹ | Sodium in | Sodiumbisulfite | 4 days/week | Sodiumbisulfite |
| | consecutive | | vehicle | in vehicle | (Vehicle + | in vehicle |
| | days/week | 12 mg/ml | 60 mg/ml | 3.85 mg/ml | Menadione | 3.85 mg/ml |
| | (full | 60 mg/Kg | 30 mg/kg | 19.25 mg/kg | only) | 19.25 mg/Kg |
| | combination | | | | | |
| 3 | 4X4 + | Cyclophosphamide | Diclofenac | Menadione | 4X4 + | Menadione |
| | Xylitol | in Xylitol- | Sodium in | Sodiumbisulfite | Xylitol | Sodiumbisulfite |
| | 2 non- | vehicle² | Xylitol- | in Xylitol- | 4 days/week | in Xylitol- |
| | consecutive | | vehicle | vehicle | (Vehicle + | vehicle |
| | days/week | 12 mg/ml | 6 mg/ml | 3.85 mg/ml | Menadione + | 3.85 mg/ml |
| | (full | 60 mg/Kg | 30 mg/kg | 19.25 mg/kg | Xylitol) | 19.25 mg/Kg |
| | combination + | | | | | |
| | xylitol) | | | | | |

¹Vehicle: 2% Pluronic, 2% Solutol HS-15 in DDW
²Xylitol-Vehicle: 2% Pluronic, 2% Solutol HS-15, 60% Xylitol in DDW

### EXAMPLE 2

### Experiments Demonstrating Efficacy of Addition of Sulfasalazine to Tiltan Formulation

As used herein and throughout, the term “Tiltan formulation” or “Tiltan” is a treatment regimen as described in group 3 (“4×4”+xylitol; full combination+xylitol) of Table 1.

In the experiments discussed below it is shown that addition of Sulfasalazine to the Tiltan formulation led to improved-tumor suppression in a murine in vivo model.

### Protocol

In the following experiments different drug combinations for suppression of tumor growth in mice were tested in vivo. The drug combinations were compared to both a control group, receiving a vehicle containing non-active ingredients only, and to a Tiltan group, receiving the current Tiltan drug combination.

Inoculation: 3.5×10⁵ cells of mouse mammary carcinoma (EMT₆/CTX) were injected subcutaneously to 7-8 week-old mice of the CB6F1 strain (a cross between Balbc and C57b1), in the center of their backs. The mice were then marked and divided into groups.

Tumor measurement: The tumor size was measured twice a week and plotted in a graph. The formula used for assessing the 3 dimensional size of the tumor was: length×width×width×0.52. The width measurement was also used as an indication for tumor height, and the 0.52 is a normalizing factor.

Injections: Mice were injected with either treatment or vehicle daily, 6 days a week. Injection volume was 0.05 mL per 10 g body weight (25 g mice received 0.125 mL). All injections were performed intraperitoneally.

Treatment composition: The experimental drugs are based on the Tiltan formulation and consistent with its regimen. The week is thus divided into two treatment types, cytotoxic and non-cytotoxic days.

On non-cytotoxic days, the mice receive the following drugs: Xylitol—60% and Menadione Sodium Bisulfite (70% purity)—27.5 mg/Kg/day.

On cytotoxic days, the following drugs are added to the previous formulation: Diclofenac Sodium—30 mg/Kg/day, Cyclophosphamide (CTX)—60 mg/Kg/day.

All drugs mentioned above are delivered in a vehicle containing Double Distilled Water (DDW), 2% Solutol HS-15 and 2% Lutrol (Pluronic) F-68.

Groups receiving Sulfasalazine are administered Sulfasalazine in addition to regular Tiltan treatment. The daily dosage is according to the experimental regimen, and ranges between 150-350 mg/Kg/day.

Preparation: For control group: DDW is added in the amount of 98% of final volume for solution. 2% Solutol (liquid) and 2% Lutrol are then added, and solution is stirred well.

For all non-Sulfasalazine containing groups: DDW volume added is 60% of final volume of solution due to Xylitol dissolving and volume increase. 60% Xylitol must be dissolved in preheated DDW (−60° C.) and stirred until solution is clear. 98% of final solution volume is measured and 2% Solutol (liquid) is then added. All other drugs are then added to the Xylitol solution and stirred until solution is homogenous.

For Sulfasalazine preparations: DDW volume added is 60% of final volume of solution due to Xylitol dissolving and volume increase. In order to increase Sulfasalazine solubility, pH must be basic, and thus Na₂CO₃ is added to DDW to a concentration of 0.2M. pH is then checked to be 10.5. Sulfasalazine is then added and the pH neutralized. Solution is then heated (−60° C.) and 60% Xylitol is added. 98% of final solution volume is measured and 2% Solutol (liquid) is then added. All other drugs are then added to the Xylitol solution and stirred until solution is homogenous.

Treatment regimen: Treatment was initiated once small tumors were visible on the majority of mice (approximately day 5 or 6 after inoculation). The first treatment is cytotoxic and marked as day 1 of the week (D1).

Cytotoxic treatment is given on day 1 and 4 of each week (D1 and D4 respectively). Non-cytotoxic treatment is given on days 2, 3, 5 and 6 (D2, D3, D5 and D6 respectively). No treatment is given on the 7th day. Sulfasalazine treatment is given either on cytotoxic or non-cytotoxic days according to experimental groups. The control group is given the vehicle every day.

Experiments TB002 and TB004 continued for 33 and 29 days following inoculation respectively.

### Experiment TB002:

The TB002 experiment includes three groups: Control, Tiltan and a group receiving Sulfasalazine treatment. Control and Tiltan groups received treatment as specified above in “Treatment regimen”. The group receiving Sulfasalazine treatment were given a dose of 350 mg/Kg/day of Sulfasalazine (SSZ) on cytotoxic days (D1 & D4), while resuming regular Tiltan treatment on non-cytotoxic days.

### Experiment TB004:

The TB004 experiment includes three groups: Control, Tiltan and a group receiving Sulfasalazine treatment. Control and Tiltan groups received treatment as specified above in “Treatment regimen”. The group receiving Sulfasalazine treatment were given a dose of 150 mg/Kg/day of Sulfasalazine on cytotoxic days (D1 & D4) and a dose of 350 mg/Kg/day of Sulfasalazine on non-cytotoxic days (D2, 3, 5 & 6).

### Results

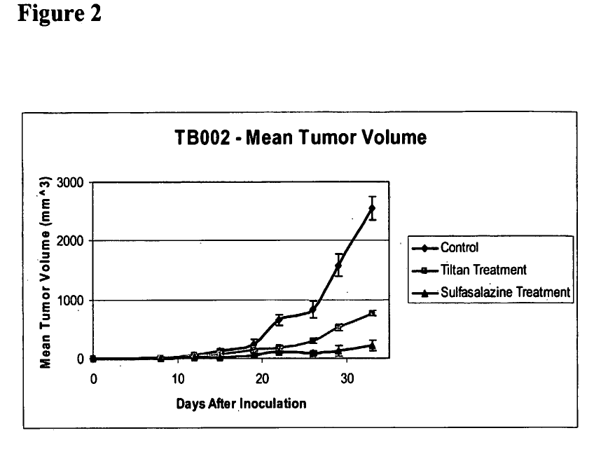

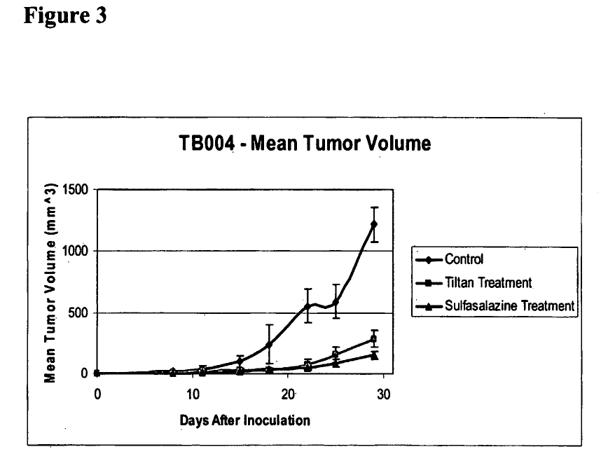

The results of both experiments are displayed in the FIGS. 2 and 3. Mean tumor volume (mm³) of control vs. Tiltan and Sulfasalazine treatment groups as a function of time after inoculation (n=7-8 mice per group; SE).

### EXAMPLE 3

Case Study: Individual with Ovarian Cancer with lung and liver metastases.

Individual is a sixty year old with ovarian cancer with lung and liver metastasis.

Time 0: Metastatic Adenocarcinoma ovary (stage 1V); TAH+BSO

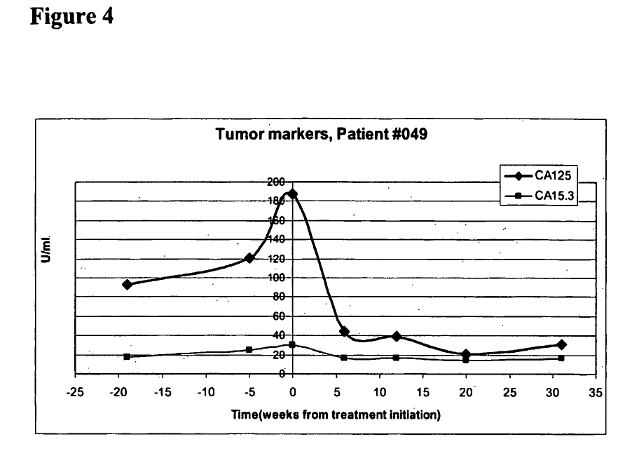

Time 0+5 months: adjuvant treatment with Carboplatin & Taxol. Following an increase in the CA-125 marker (see FIG. 4), the patient was referred to the TiltAn treatment (Performance status—0).

Time 0+30 months: Initiation of TiltAn treatment (50% dose wks 1+2; 75% dose wks 3+4; full dose wks 5 and on).

The following is a description of the full dose treatment: Dose administered twice a week on Days 1 and 4 of a weekly cycle of treatment:

50 ml aqueous solution of 60% Xylitol that contains the following agents:

| | |
|---|---|
| Cyclophosphamide | 400 mg |
| Diclofenac | 200 mg |
| Vitamin K3 | 140 mg |

Dose administered five times a week on Days 2, 3, 5, 6 and 7 of a weekly cycle of treatment:

50 ml aqueous solution of 60% Xylitol that contains:
Vitamin K3→ 140 mg

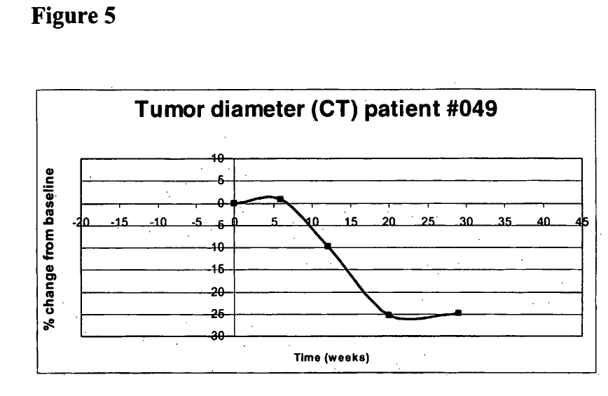

Results (for details see FIGS. 4 and 5):

Tumor markers: Following treatment initiation, both tumor markers CA-125 and CA-15.3 dropped to the normal range level and stayed at this range through wk 30.

Tumor size: After 6 wks on the TiltAn treatment, a CT of the pelvis, abdomen and thorax revealed stable disease. On wk 12 there was a decrease in the dimensions of the liver metastasis and this decrease proceeded through wk 30.

What is claimed is:

1. A pharmaceutical composition comprising a combination of at least one angiogenesis inhibitor, at least one agent that enhances accumulation of intracellular NADH+H⁺ and a pharmaceutically acceptable carrier.

2. The pharmaceutical composition of claim 1, wherein the at least one angiogenesis inhibitor is selected from the group consisting of a direct angiogenesis inhibitor, an indirect angiogenesis inhibitor, a cytotoxic agent, and an inhibitor of pro-angiogenic growth factors.

3. The pharmaceutical composition of claim 1, wherein the composition further comprises at least one anti-inflammatory agent and a redox quinone.

4. The pharmaceutical composition of claim 2, wherein the direct angiogenesis inhibitor is selected from the group consisting of Angiostatin, Bevacizumab (Avastin), Arresten, Canstatin, Combretastatin, Endostatin, NM-3, Thrombospondin, Tumstatin, 2-methoxyestradiol, and Vitaxin.

5. The pharmaceutical composition of claim 2, wherein the indirect angiogenesis inhibitor is selected from the group consisting of ZD1839 (Iressa), ZD6474, OS1774 (Tarceva), C11033, PK11666, IMC225 (Erbitux), PTK787, SU6668, SU11248, Herceptin, TNP-470, HPMA copolymer-TNP-470 and IFN-α.

6. The pharmaceutical composition of claim 2, wherein the cytotoxic agent is selected from the group consisting of cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, and paclitaxel.

7. The pharmaceutical composition of claim 2, wherein the cytotoxic agent is cyclophosphamide or ifosfamide.

8. The pharmaceutical composition of claim 2, wherein the inhibitors of pro-angiogenic growth factors are selected from the group consisting of anti-VEGF, anti-VEGF-receptor antibodies, and inhibitors of the protein-kinase domain of VEGF-R, FGF-R or PDGF-R.

9. The pharmaceutical composition of claim 2, wherein the at least one anti-inflammatory agent is selected from the group consisting of steroidal drugs (such as dexamethasone), non-steroidal anti-inflammatory agents, including COX 1-2 inhibitors and NFκB inhibitors.

10. The method of claim 9, wherein the NFκB inhibitor is sulfasalazine.

11. The method of claim 9, wherein the NFκB inhibitor is selected from the group consisting of sulfasalazine, 5-aminosalicylate, and sulfapyridine.

12. The pharmaceutical composition of claim 3, wherein the at least one anti-inflammatory agent is diclofenac, indomethacin and/or sulfasalazine.

13. The pharmaceutical composition of claim 3, wherein the redox quinone is Vitamin K₃ (menadione or menadione sodiumbisulfite).

14. The pharmaceutical composition of claim 1, wherein the at least one angiogenesis inhibitor comprises a cytotoxic agent, a COX 1-2 inhibitor (diclofenac or indomethacin), a redox quinone (Vitamin K₃, menadione or menadione sodiumbisulfite) and a pharmaceutically acceptable carrier.

15. The pharmaceutical composition of claim 12, wherein the at least one angiogenesis inhibitor further comprises an NFκB inhibitor (sulfasalazine).

16. The pharmaceutical composition of claim 1, wherein the agent that enhances intracellular accumulation of NADH+H⁺ is a poly-alcohol.

17. The pharmaceutical composition of claim 16, wherein the poly-alcohol is xylitol.

18. The pharmaceutical composition of claim 16, wherein the poly-alcohol is selected from the group consisting of xylitol, mannitol, sorbitol, arabinol, and iditol.

19. The pharmaceutical composition of claim 1 further comprising an inhibitor of MMP.

20. The pharmaceutical composition of claims 1, wherein the at least one angiogenesis inhibitor, poly-alcohol and the pharmaceutically acceptable carrier are formulated as an aqueous suspension or solution.

21. The pharmaceutical composition of claim 1, wherein the composition is formulated for oral administration.

22. The pharmaceutical composition of claim 21, wherein at least one component of said composition is supplied in a dry form and reconstituted prior to oral administration.

23. The pharmaceutical composition of claim 21, wherein the composition further contains a flavoring agent.

24. The pharmaceutical composition of claim 23, wherein flavoring agent is menthol and/or anethol.

25. The pharmaceutical composition of claims 1, wherein the at least one angiogenesis inhibitor, poly-alcohol and the pharmaceutically acceptable carrier are administered on non-consecutive days while at the alternate days only poly-alcohol, a redox quinone and the pharmaceutically acceptable carrier are administered.

26. The pharmaceutical composition of claims 25, wherein the at least one angiogenesis inhibitor, poly-alcohol and the pharmaceutically acceptable carrier are administered twice a week and the poly-alcohol, a redox quinone and the pharmaceutically acceptable carrier are administered daily during the rest of the week.

27. A method of inhibiting angiogenesis in a tissue of a mammal having an angiogenic disease or disorder or at risk for developing an angiogenic disease or disorder comprising: administering to a tissue an angiogenesis-inhibiting amount of at least one angiogenesis inhibitor, at least one agent that enhances intracellular accumulation of NADH+H⁺ and a pharmaceutically acceptable carrier.

28. The method of claim 27, further comprising administering an inhibitor of MMP.

29. The method of claim 27, wherein the angiogenic disease or disorder is cancer.

30. The method of claim 27, wherein the cancer is selected from the group consisting of lung cancer (e.g. adenocarcinoma and including non-small cell lung cancer), pancreatic cancers (e.g. pancreatic carcinoma such as, for example exocrine pancreatic carcinoma), colon cancers (e.g. colorectal carcinomas, such as, for example, colon adenocarcinoma and colon adenoma), prostate cancer including the advanced disease, hematopoietic tumors of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumors of mesenchymal origin (e.g. fibrosarcomas and rhabdomyosarcomas), melanomas, teratocarcinomas, neuroblastomas, gliomas, benign tumor of the skin (e.g. keratoacanthomas), breast carcinoma (e.g. advanced breast cancer), kidney carcinoma, ovary carcinoma, bladder carcinoma and epidermal carcinoma.

31. The method of claim 27, wherein the angiogenic disease or disorder is macular degeneration, obesity, retinopathy, diabetic retinopathy, arthritis, rheumatoid arthritis, psoriasis and restenosis.

32. The method of claim 27, wherein the composition is formulated as an aqueous suspension or a solution.

33. The method of claim 27, wherein said administering comprises intravenous, transdermal, intrasynovial, intramuscular, or oral administration.

34. The method of claims 27, wherein the mammal is selected from the group consisting of a human, cat, dog or horse.

35. The method of claim 27, wherein said administration is twice a week on days 1 and 4 of a weekly cycle of treatment and wherein 25-100 ml aqueous solution of 30-60% Xylitol that contains Cyclophosphamide at about 200-600 mg, Diclofenac at about 100-300 mg, and Vitamin K₃ at about 100-500 mg is administered.

36. The method of claim 27, wherein said administration is twice a week on days 1 and 4 of a weekly cycle of treatment and wherein 25-100 ml aqueous solution of 30-60% Xylitol that contains Cyclophosphamide at about 200-600 mg, Diclofenac at about 100-300 mg, Vitamin K3 at about 100-500 mg and Sulfasalazine at about 500-3,000 mg is administered.

37. The method of claim 27, wherein said administration is administered five times a week on Days 2, 3, 5, 6 and 7 of a weekly cycle of treatment and wherein 25-100 ml aqueous solution of 30-60% Xylitol that contains Vitamin K3 at about 100-500 mg is administered.

38. The method of claim 27, wherein said administration is administered five times a week on Days 2, 3, 5, 6 and 7 of a weekly cycle of treatment and wherein 25-100 ml aqueous solution of 30-60% Xylitol that contains Vitamin K3 at about 100-500 mg and Sulfasalazine at about 200-1,000 mg is administered.

39. A kit for the treatment or prevention of angiogenic disease or disorder comprising separate vials containing an angiogenesis inhibitor, an agent that enhances intracellular-accumulation of NADH+H⁺ together with a redox quinone, a pharmaceutically acceptable carrier and directions for administration of each component.

40. The pharmaceutical composition of claim 9, wherein the COX-2 inhibitor is diclofenac or indomethacin.
